(19) United States
(12) Patent Application Publication
(10) Pub. No.: US 2026/0035377 A1
(43) Pub. Date: Feb. 5, 2026

(54) AMIDE-BASED MOLECULAR CAGES FOR THE HIGHLY SELECTIVE AND SENSITIVE DETECTION OF NICOTINE

(71) Applicant: University of South Florida, Tampa, FL (US)

(72) Inventors: Ioannis Spanopoulos, Tampa, FL (US); Mina Bagherifard Sharabiani, Tampa, FL (US); Amrit Kaur, Tampa, FL (US)

(21) Appl. No.: 19/288,618

(22) Filed: Aug. 1, 2025

Related U.S. Application Data
(60) Provisional application No. 63/678,844, filed on Aug. 2, 2024.

(51) Int. Cl. C07D 487/08; G01N 21/66; G01N 33/18; G01N 33/24; G01N 33/52; G01N 33/94

(52) U.S. Cl. CPC C07D 487/08; G01N 21/66; G01N 33/1826; G01N 33/24; G01N 33/52; G01N 33/94

(57) ABSTRACT

Disclosed are amide-based molecular cages and methods of making them and using them to detect nicotine, cotinine, and other pyridine-containing compounds of similar structure. In a specific example, compounds of Formula I are disclosed:

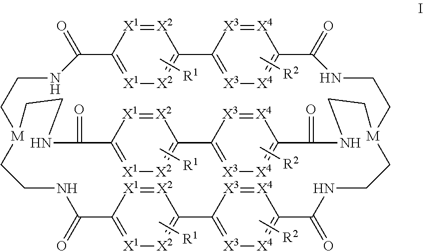

wherein
each X¹, X², X³, and X⁴ are, independently, CH, N, CR¹, or CR²;
   R¹ and R² are, independently, H, OH, SH, C₁₋₆ alkyl, C₁₋₆ alkoxyl, C₁₋₆ thioalkyl C₁₋₆ alkyl-SO₄, F, Cl, Br, NH₂, NO₂, or CN; and
M is N or N⁺R⁴, wherein R⁴ is H or C₁₋₆ alkyl.

### CROSS REFERENCE TO RELATED APPLICATIONS

This application claims the benefit of priority to U.S. Provisional Application 63/678,844, filed on Aug. 2, 2024, which is incorporated herein by reference in its entirety.

### BACKGROUND

Nicotine is the primary alkaloid found in the tobacco plant, featuring a major role in tobacco addiction (B. Le Foll, et al., Nat Rev Dis Primers, 2022, 8, 19). The human body can be exposed to nicotine via active and passive smoking of cigarettes (including e-cigarettes), cigars, and other tobacco-based products (P. Marques, et al., Respir. Res., 2021, 22, 151; L. S. Flor, et al., Nat. Med., 2024, 30, 149-167). The consumption of nicotine has adverse effects on human health and can promote the development of cardiovascular diseases, cancer, Alzheimer's disease, Parkinson's disease, Tourette syndrome, and various respiratory disorders (B. Lin, et al., ACS Sens., 2019, 4, 1844-1850; P. A. Newhouse, et al., Biol. Psychiatry, 2001, 49, 268-278; A. Alhowail, Mol. Med. Rep., 2021, 23, 1-6; G. K. Lloyd, et al., J Pharmacol Exp Ther, 2000, 292, 461-467). It has been reported that as little as 0.5-1 mg of nicotine per kg of body weight in adults can be fatal (S. Mondal, et al., ACS Appl. Bio Mater., 2024, 7, 2346-2353). Considering the above, it is of paramount importance to develop efficient, selective, and reliable methods for the facile detection and monitoring of nicotine in human fluids, such as urine, saliva, and serum (Z. Hu, et al., J. Mater. Chem. A, 2023, 11, 4739-4750; A. N. Ramdzan, et al., TrAC, Trends Anal. Chem., 2018, 105, 89-97; F. Soleimani, et al., Sci. Total Environ., 2022, 813, 152667).

To this end, several analytical methods have been developed for detecting nicotine, such as gas chromatography (GC), high-performance liquid chromatography (HPLC), radioimmunoassay, capillary electrophoresis, electrochemiluminescence (ECL), and surface-enhanced Raman spectroscopy (SERS) (O. Gould, et al., Chemosensors, 2023, 11, 527; A. Marsh, et al., Electrophoresis, 2004, 25, 1270-1278; A. Karthika, et al., Ultrason. Sonochem., 2019, 55, 196-206; B. Lin, et al., ACS Appl. Mater. Interfaces, 2021, 13, 37638-37644). HPLC is the most frequently employed technique for the quantitative analysis of nicotine in biological samples (M. Yasuda, et al., J. Chromatogr. B, 2013, 934, 41-45). Despite their efficiency, the aforementioned techniques require intricate pretreatment protocols, costly and heavy equipment, and highly skilled trained operators (A. E.-G. E. Amr, et al., ACS Omega, 2021, 6, 11340-11347). Therefore, emerging technologies that enable facile, highly selective, and sensitive nicotine detection without the use of expensive and complex equipment are greatly sought after, especially for portable applications. Evidently, fluorescence-based detection is rapid, non-invasive, selective, and highly sensitive, enabling easy real-time monitoring without a lengthy sample preparation process (K. Liu, et al., Chem. Commun., 2019, 55, 12679-12682). Consequently, a tailor-made fluorescence-based nicotine sensor poses an excellent choice for detecting trace amounts of nicotine in various samples, such as e-liquids, wastewater, and biological fluids, to name a few (N. Cennamo, et al., Sens Actuators B Chem., 2014, 191, 529-536; J. Wu, et al., (′hem. Soc. Rev., 2011, 40, 3483).

To date, various fluorescence-based probes, such as metal-organic frameworks (MOFs), carbon nanodots, and metallo-porphyrins have been developed to detect nicotine (D. Yan, et al., ACS Appl. Mater. Interfaces, 2019, 11, 47253-47258; X. Xu, et al., Microchem. J., 2021, 160, 105708; G. R. Deviprasad, et al., Chem. Commun., 2000, 1915-1916). Although these probes have several advantages as tools for molecular recognition, their complex and expensive synthesis and composition of toxic elements limit their widespread utilization (Z. Hu, et al., J. Mater. Chem. A, 2023, 11, 4739-4750; C. Yang, et al., Inorg. Chem., 2023, 62, 20458-20466). Notably, fully organic molecular cages have garnered significant research interest due to their wide applications in gas storage, catalysis, sensing, and guest binding (S. La Cognata, et al., Chem. Commun., 2023, 59, 13668-13678). In recent years, the development of water-soluble cages based on pyridinium cations, acyl hydrazone polyamides, urea cages, heterodimeric disulfide cages, imidazolium cages, and imine cages has enabled them to be widely applied in the field of molecular recognition (W. Liu, et al., JACS, 2021, 143, 15688-15700; H. Li, H. Zhang, et al., Nat. Chem., 2015, 7, 1003-1008). Amide cages are widely used for the molecular recognition of biomolecules such as carbohydrates, amino acids, and amines (K. G. Andrews, et al., Chem. Eur. J., 2023, 29, 26; L. Tapia, et al., Org. Biomol. Chem., 2021, 19, 9527-9540; X. Yang, et al., Chem. Rev., 2023, 123, 4602-4634; J. C. Lauer, et al., Chem. Eur. J., 2022, 28; S. Maji, et al., (hem. Eur. J., 2024, 30, 1-9). Even though significant progress has been made, the majority of reported methods—particularly those involving irreversible bonds—entail challenging features such as template-based synthesis, multistep reactions, limited yields, and laborious purification methods (Id.). These characteristics hinder their widespread utilization for scalable real-life applications. What is thus needed are new compositions and methods for the detection of nicotine. The compositions and methods disclosed herein address these and other needs.

### SUMMARY

Disclosed are compounds, compositions, and methods of making and using thereof. In one aspect, the disclosed subject matter relates to amide-based cages and methods of making them and using them to detect nicotine, cotinine, and other pyridine-containing compounds of similar structure.

In a specific aspect, disclosed are compounds of Formula I,

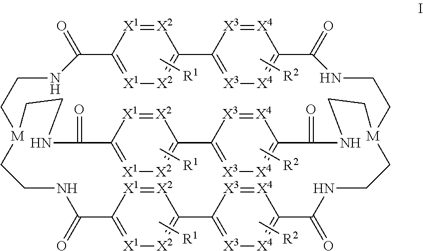

wherein
   each X¹, X², X³, and X⁴ are, independently, CH, N, CR¹, or CR²;
   R¹ and R² are, independently, H, OH, SH, C₁₋₆ alkyl, C₁₋₆ alkoxyl, C₁₋₆ thioalkyl C₁₋₆ alkyl-SO₄, F, Cl, Br, NH₂, NO₂, or CN; and
   M is N or N⁺R⁴, wherein R⁴ is H or C₁₋₆ alkyl.

Also disclosed are compounds of Formulas II-IV as described herein.

Additional advantages of the disclosed subject matter will be set forth in part in the description that follows and the figures, and in part will be obvious from the description or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 shows the synthetic route of the BiP-Am cage: (a) 4.8 mmol of [1,1′-Biphenyl]-4,4′-dicarbaldehyde, 3.2 mmol of Tris(2-aminoethyl) amine, 275 mL of ACN, 0° C., 7 days. (b) 0.6 mmol of Schiff base, 20.1 mmol of Sodium chlorite, 7.4 mmol of Ammonium chloride, and 36.8 mmol of (1S)-(−)-α-pinene, 100 mL of dry THF under reflux for 48 hours. (c) The interaction of the BiP-Am probe with nicotine solution results in a sharp decrease in the intensity of the emission spectra when excited at 273 nm.

FIG. 2 shows emission spectra evolution of the BiP-Am probe via successively adding 0.1 mM nicotine solution to a H₂O:DMSO (9.9:0.1, v:v, 10 μM) sample.

FIG. 3A shows the PL quenching efficiency of BiP-Am probe (H₂O:DMSO (9.9:0.1, v:v, 10 μM)) upon addition of 0.1 mM Nicotine solution into 3 mL probe solution. FIG. 3B shows a calibration curve for BiP-Am probe response to the addition of 0.1 mM Nicotine solution. FIG. 3C shows the emission intensity of pure BiP-Am in H₂O:DMSO (9.9:0.1, v:v, 10 μM) (light-gray) and its response upon the addition of various interferents (dark-gray and black columns) are presented. Nicotine significantly quenched the BiP-Am probe emission.

FIGS. 4A-4B show SEM images of (FIG. 4A) the BiP-Am sample at 100 μm scale and (FIG. 4B) the BiP-Am-Nicotine aggregate at 30 μm scale.

FIG. 5A (absorption) and FIG. 5B (emission) are spectra of the BiP-Am probe under excitation at 273 nm in various solvents (solvent: DMSO (9.9:0.1, v:v, 10 μM)).

FIG. 6A (absorption) and FIG. 6B (emission) are spectra of the BiP-Am probe under excitation at 273 nm upon varying water fraction in DMSO from 0-99% by volume; H₂O:DMSO (9.9:0.1, v:v, 10 μM) shows the most enhanced peak in both absorption and emission spectra.

FIG. 7 shows the UV-Vis spectrum of nicotine (trace A; 0.1 M), BiP-Am probe in H₂O:DMSO (trace B; 9.9:0.1, v:v, 10 μM), and the change in absorbance after the addition of nicotine (trace C; 10 μL of 1 mM solution) to the BiP-Am probe in H₂O:DMSO (9.9:0.1, v:v, 10 μM).

FIG. 8 shows a selectivity assessment of the BiP-Am probe in H₂O:DMSO (9.9:0.1, v/v, 10 μM). The light-gray represents the emission intensity of pure BiP-Am, while the dark-gray columns depict the changes in emission upon the addition of various interfering analytes. The black columns represent the emission intensity in the presence of both the interferent and nicotine. All emission spectra were recorded under excitation at 273 nm.

FIG. 9 is a Stern-Volmer diagram of BiP-Am in H₂O:DMSO (9.9:0.1, v:v, 10 μM) upon addition of 0.1 M Nicotine to the solution.

FIG. 10 shows the time-resolved photoluminescence decay of the BiP-Am before and after the addition of nicotine to the sample in H₂O:DMSO (9.9:0.1, v:v, 10 μM).

FIG. 11 shows ¹H NMR spectra of BiP-Am, BiP-Am-Nicotine and Nicotine in DMSO-d₆:D₂O (9:1, v:v).

FIGS. 12A-12B show DLS data of (FIG. 12A) BiP-Am and (FIG. 12B) after the addition of nicotine to BiP-Am probe in H₂O:DMSO (9.9:0.1, v:v, 10 μM).

FIG. 13 shows the emission spectra of probe BiP-Am-Nicotine in H₂O:DMSO (9.9:0.1, v:v, 10 μM) under various excitation wavelengths (263-313 nm) (P. Sharma, et al., ACS Omega, 2020, 5, 19654-19660).

FIG. 14A-14B show normalized absorption (FIG. 14A) and emission (FIG. 14B) spectra of Nicotine and BiP-Am in H₂O:DMSO (9.9:0.1, v:v, 10 μM) under excitation at 263 nm and 273 nm, respectively.

FIG. 15 shows the emission spectra of BiP-Am in H₂O:DMSO (9.9:0.1, v:v, 10 μM) under excitation at 273 nm (black); after the addition of 50 μL of prepared cigarette solution (light-gray); and after the addition of 0.5 mM Nicotine (dark-gray).

FIG. 16 shows the emission spectra of BiP-Am in H₂O:DMSO (9.9:0.1, v:v, 10 μM) under excitation at 273 nm (black); after the addition of 50 μL of prepared urine solution (light-gray); and after the addition of 0.5 mM Nicotine (dark-gray).

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

As can be apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### General Definitions

In this specification and in the claims that follow, reference will be made to many terms, which shall be defined to have the following meanings:

As used herein, “comprising” is to be interpreted as specifying the presence of the stated features, integers, steps, or components as referred to but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an,” and “the” include plural referents unless the context clearly dictates otherwise.

“Optional” or “optionally” means that the subsequently described event or circumstance can or cannot occur and that the description includes instances where the event or circumstance occurs and instances where it does not.

### Chemical Definitions

A weight percent of a component, unless specifically stated to the contrary, is based on the total weight of the formulation or composition in which the component is included.

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Compounds described herein may contain one or more double bonds and, thus, potentially give rise to cis/trans (E/Z) isomers, as well as other conformational isomers. Unless stated to the contrary, all such possible isomers are contemplated, as well as mixtures of such isomers.

Compounds described herein may also present as an equilibrium of tautomers. For example, ketones with an α-hydrogen can exist in an equilibrium of the keto form and the enol form. Likewise, amides with an N-hydrogen can exist in an equilibrium of the amide form and the imidic acid form. Unless stated to the contrary, all possible tautomers of the compounds described herein are contemplated.

### Compounds

In one aspect, disclosed herein are compounds of Formula I:

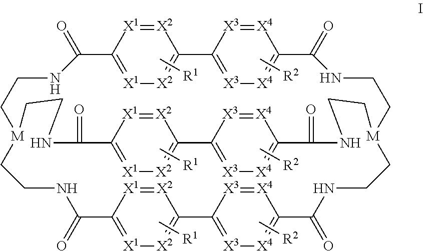

wherein
   each X¹, X², X³, and X⁴ are, independently, CH, N, CR¹, or CR²;
   R¹ and R² are, independently, H, OH, SH, C₁₋₆ alkyl, C₁₋₆ alkoxyl, C₁₋₆ thioalkyl, C₁₋₆ alkyl-SO₄, F, Cl, Br, NH₂, NO₂, or CN; and
   each M is N or N⁺R⁴, wherein R⁴ is H or C₁₋₆ alkyl.

Disclosed herein in another aspect are compounds of Formula II:

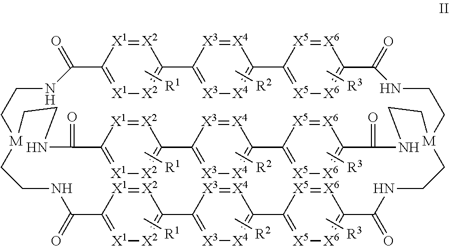

wherein
   each X¹, X², X³, X⁴, X³, and X⁶ are, independently, CH, N, CR¹, CR², or CR³;
   R¹, R², and R³ are, independently, H, OH, SH, C₁₋₆ alkyl, C₁₋₆ alkoxyl, C₁₋₆ thioalkyl, C₁₋₆ alkyl-SO₄, F, Cl, Br, NH₂, NO₂, or CN; and
   each M is N or N⁺R⁴, wherein R⁴ is H or C₁₋₆ alkyl.

Disclosed herein in still another aspect are compounds of Formula III:

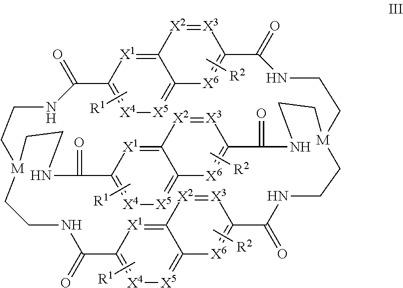

wherein
   each X¹, X², X³, X⁴, X⁵, and X⁶ are, independently, CH, N, CR¹, or CR²;
   R¹ and R² are, independently, H, OH, SH, C₁₋₆ alkyl, C₁₋₆ alkoxyl, C₁₋₆ thioalkyl, C₁₋₆ alkyl-SO₄, F, Cl, Br, NH₂, NO₂, or CN; and
   each M is N or N⁺R⁴, wherein R⁴ is H or C₁₋₆ alkyl.

Disclosed herein in still another aspect are compounds of Formula IV:

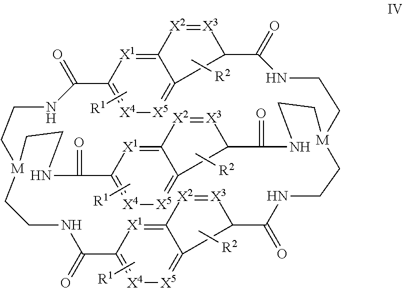

wherein
   each X¹, X², X³, X⁴, and X⁵ are, independently, CH, N, CR¹, or CR²;
   R¹ and R² are, independently, H, OH, SH, C₁₋₆ alkyl, C₁₋₆ alkoxyl, C₁₋₆ thioalkyl, C₁₋₆ alkyl-SO₄, F, Cl, Br, NH₂, NO₂, or CN; and
   each M is N or N⁺R⁴, wherein R⁴ is H or C₁₋₆ alkyl.

In a specific example, disclosed is a compound of Formula I, wherein each of X¹, X², X³, and X⁴ are CH, and each of R¹ and R² are H, and M is N, which is shown below and is also referred to herein as BiP-Am:

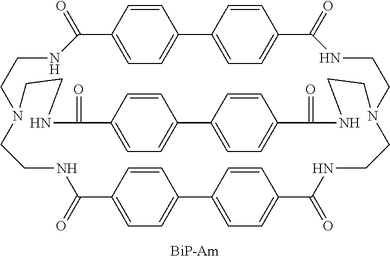 [SMILES: O=C1NCCN2CCNC(=O)c3ccc(cc3)-c3ccc(cc3)C(=O)NCCN(CCNC(=O)c3ccc(cc3)-c3ccc1cc3)CCNC(=O)c1ccc(cc1)-c1ccc(cc1)C(=O)NCC2 ; "BiP-Am"]

In another example, disclosed is a compound of Formula I, wherein any one of X¹, X², X³, and X⁴ is, independently, N. In another example, disclosed is a compound of Formula I, wherein any one or more of X¹, X², X³, and X⁴ is, independently, N. In another example, disclosed is a compound of Formula I, wherein one X¹ is N. In another example, disclosed is a compound of Formula I, wherein one X² is N. In another example, disclosed is a compound of Formula I, wherein one X³ is N. In another example, disclosed is a compound of Formula I, wherein one X⁴ is N. The selections in this paragraph can be made independently of one another.

In another example, disclosed is a compound of Formula I, wherein any two X¹, X², X³, and X⁴ are, independently, N. In another example, disclosed is a compound of Formula I, wherein any two X¹'s are N. In another example, disclosed is a compound of Formula I, wherein any two X²'s are N. In another example, disclosed is a compound of Formula I, wherein any two X³'s are N. In another example, disclosed is a compound of Formula I, wherein any two X⁴'s are N. The selections in this paragraph can be made independently of one another.

In another example, disclosed is a compound of Formula I, wherein any three X¹, X², X³, and X⁴ are N. In another example, disclosed is a compound of Formula I, wherein any three X¹'s are N. In another example, disclosed is a compound of Formula I, wherein any three X²'s are N. In another example, disclosed is a compound of Formula I, wherein any three X³'s are N. In another example, disclosed is a compound of Formula I, wherein any three X⁴'s are N. The selections in this paragraph can be made independently of one another.

In another example, disclosed is a compound of Formula I, wherein one of R¹ and R² is OH, SH, C₁₋₆ alkyl (e.g., CH₃, C₂H₅, C₃H₇), C₁₋₆ alkoxyl (e.g., OCH₃, OC₂H₅, OC₃H₇), C₁₋₆ thioalkyl, C₁₋₆ alkyl-SO₄, F, Cl, Br, NH₂, NO₂, or CN. In still other examples, one of R¹ and R² is OH. In still other examples, one of R¹ and R² is SH. In still other examples, one of R¹ and R² is OCH₃. In still other examples, one of R¹ and R² is SCH₃. In still other examples, one of R¹ and R² is CH₃. In still other examples, one of R¹ and R² is OC₂H₅. In still other examples, one of R¹ and R² is SC₂H₅. In still other examples, one of R¹ and R² is C₂H₅. In still other examples, one of R¹ and R² is F. In still other examples, one of R¹ and R² is Cl. In still other examples, one of R¹ and R² is Br. In still other examples, one of R¹ and R² is NH₂. In still other examples, one of R¹ and R² is NO₂. In still other examples, one of R¹ and R² is CN. The selections in this paragraph can be made independently of one another.

In another example, disclosed is a compound of Formula I, wherein both of R¹ and R² are OH, SH, C₁₋₆ alkyl (e.g., CH₃, C₂H₅, C₃H₇), C₁₋₆ alkoxyl (e.g., OCH₃, OC₂H₅, OC₃H₇), C₁-6 thioalkyl, C₁₋₆ alkyl-SO₄, F, Cl, Br, NH₂, NO₂, or CN. In still other examples, both of R¹ and R² are OH. In still other examples, both of R¹ and R² are SH. In still other examples, both of R¹ and R² are OCH₃. In still other examples, both of R¹ and R² are SCH₃. In still other examples, both of R¹ and R² are CH₃. In still other examples, both of R¹ and R² are OC₂H₅. In still other examples, both of R¹ and R² are SC₂H₅. In still other examples, both of R¹ and R² are C₂H₅. In still other examples, both of R¹ and R² are F. In still other examples, both of R¹ and R² are Cl. In still other examples, both of R¹ and R² are Br. In still other examples, both of R¹ and R² are NH₂. In still other examples, both of R¹ and R² are NO₂. In still other examples, both of R¹ and R² are CN.

In another example, disclosed is a compound of Formula I, wherein each Mis N. In another example, disclosed is a compound of Formula I, wherein each Mis N⁺R⁴, with R⁴ being methyl, ethyl, propyl, isopropyl, butyl, sec-butyl, tert-butyl, pentyl, or hexyl. In a specific example, R⁴ is methyl. In another example, disclosed is a compound of Formula I, wherein each M is N⁺R⁴, with R⁴ being hydrogen. In another example, disclosed is a compound of Formula I, wherein one M is N and the other Mis N⁺R⁴, with R⁴ being H or C₁-C₆ alkyl.

In another example, disclosed is a compound of any of Formulas II-IV, wherein any one of X¹, X², X³, X⁴, X⁵, and X⁶ is N. In another example, disclosed is a compound of Formula I, wherein any two of X¹, X², X³, X⁴, X⁵, and X⁶ are N. In another example, disclosed is a compound of Formula I, wherein any three of X¹, X², X³, X⁴, X⁵, and X⁶ are N. In another example, disclosed is a compound of Formula I, wherein any four of X¹, X², X³, X⁴, X⁵, and X⁶ are N. In another example, disclosed is a compound of Formula I, wherein any five of X¹, X², X³, X⁴, X⁵, and X⁶ are N.

In another example, disclosed is a compound of any of Formulas II-IV, wherein any one or more of X¹, X², X³, X⁴, X⁵, and X⁶ is N. In another example, disclosed is a compound of any of Formulas II-IV, wherein one X¹ is N. In another example, disclosed is a compound of any of Formulas II-IV, wherein one X² is N. In another example, disclosed is a compound of any of Formulas II-IV, wherein one X³ is N. In another example, disclosed is a compound of Formulas II-IV, wherein one X⁴ is N. In another example, disclosed is a compound of Formulas II-IV, wherein one X⁵ is N. In another example, disclosed is a compound of Formulas II-IV, wherein one X⁶ is N. The selections in this paragraph can be made independently of one another.

In another example, disclosed is a compound of any of Formulas II-IV, wherein any two X¹, X², X³, and X⁴ are N. In another example, disclosed is a compound of any of Formulas II-IV, wherein any two X¹'s are N. In another example, disclosed is a compound of any of Formulas II-IV, wherein any two X²'s are N. In another example, disclosed is a compound of any of Formulas II-IV, wherein any two X³'s are N. In another example, disclosed is a compound of any of Formulas II-IV, wherein any two X⁴'s are N. In another example, disclosed is a compound of any of Formulas II-IV, wherein any two X⁵'s are N. In another example, disclosed is a compound of any of Formulas II-IV, wherein any two X⁶'s are N. The selections in this paragraph can be made independently of one another.

In another example, disclosed is a compound of any of Formulas II-IV, wherein any three X¹, X², X³, and X⁴ are N. In another example, disclosed is a compound of any of Formulas II-IV, wherein any three X¹'s are N. In another example, disclosed is a compound of any of Formulas II-IV, wherein any three X²'s are N. In another example, disclosed is a compound of any of Formulas II-IV, wherein any three X³'s are N. In another example, disclosed is a compound of any of Formulas II-IV, wherein any three X⁴'s are N. In another example, disclosed is a compound of any of Formulas II-IV, wherein any three X⁵'s are N. In another example, disclosed is a compound of any of Formulas II-IV, wherein any three X⁶'s are N. The selections in this paragraph can be made independently of one another. In a specific example, disclosed are compounds of any of Formulas II-IV where each of R¹, R², and R³ are H.

In another example, disclosed is a compound of any of Formulas II-IV, wherein one or two of R¹, R², and R³ is OH, SH, C₁₋₆ alkyl, C₁₋₆ alkoxyl, C₁₋₆ thioalkyl, C₁₋₆ alkyl-SO₄, F, Cl, Br, NH₂, NO₂, or CN. In still other examples, one or two of R¹, R², and R³ is OH. In still other examples, one or two of R¹, R², and R³ is SH. In still other examples, one or two of R¹, R², and R³ is OCH₃. In still other examples, one or two of R¹, R², and R³ is SCH₃. In still other examples, one or two of R¹, R², and R³ is CH₃. In still other examples, one or two of R¹, R², and R³ is OC₂H₅. In still other examples, one or two of R¹, R², and R³ is SC₂H₅. In still other examples, one or two of R¹, R², and R³ is C₂H₅. In still other examples, one or two of R¹, R², and R³ is F. In still other examples, one or two of R¹, R², and R³ is Cl. In still other examples, one or two of R¹, R², and R³ is Br. In still other examples, one or two of R¹, R², and R³ is NH₂. In still other examples, one or two of R¹, R², and R³ is NO₂. In still other examples, one or two of R¹, R², and R³ is CN. The selections in this paragraph can be made independently of one another.

In another example, disclosed is a compound of any of Formulas I-IV, wherein all of R¹, R², and R³ are OH, SH, C₁₋₆ alkyl, C₁₋₆ alkoxyl, C₁₋₆ thioalkyl, C₁₋₆ alkyl-SO₄, F, Cl, Br, NH₂, NO₂, or CN. In still other examples, all of R¹, R², and R³ are OH. In still other examples, all of R¹, R², and R³ are SH. In still other examples, all of R¹, R², and R³ are OCH₃. In still other examples, all of R¹, R², and R³ are SCH₃. In still other examples, all of R¹, R², and R³ are CH₃. In still other examples, all of R¹, R², and R³ are OC₂H₅. In still other examples, all of R¹, R², and R³ are SC₂H₅. In still other examples, all of R¹, R², and R³ are C₂H₅. In still other examples, all of R¹, R², and R³ are F. In still other examples, all of R¹, R², and R³ are Cl. In still other examples, all of R¹, R², and R³ are Br. In still other examples, all of R¹, R², and R³ are NH₂. In still other examples, all of R¹, R², and R³ are NO₂. In still other examples, all of R¹, R², and R³ are CN.

In another example, disclosed is a compound of any of Formulas II-IV, wherein each M is N. In another example, disclosed is a compound of any of Formulas II-IV, wherein each Mis N⁺R⁴, with R⁴ being methyl, ethyl, propyl, isopropyl, butyl, sec-butyl, tert-butyl, pentyl, or hexyl. In a specific example, R⁴ is methyl. In another example, disclosed is a compound of Formula I-IV, wherein each M is N⁺R⁴, with R⁴ being hydrogen. In another example, disclosed is a compound of Formula I, wherein one M is N and the other M is N⁺R⁴, with R⁴ being H or C₁-C₆ alkyl.

It is expected that in the disclosed compounds introduction of multiple amide functional groups (six per molecule) coupled with aromatic moieties allows the compound to interact with an analyte (e.g., nicotine or cotinine). Evidently, the developed amide cage BiP-Am, exhibited aggregation-induced emission enhancement (AIEE) under light activation. The self-assembled molecules of BiP-Am exhibited a “turn-off” response towards nicotine in aqueous media. The probe BiP-Am exhibits nicotine-induced quenching in emission. The developed probe BiP-Am can efficiently detect nicotine in aqueous media with a limit of detection as low as 0.4 nM. Moreover, BiP-Am was deployed for real-time detection of nicotine in human urine samples and extracted commercial cigarette contents in an aqueous medium. This is the first time a molecular cage has been utilized for nicotine detection.

In other examples, disclosed are compounds of any one of Formulas I-IV and a biological or environmental sample. In other examples, disclosed are compounds of any one of Formulas I-IV and nicotine and/or cotinine. In other examples, disclosed are compounds of any one of Formulas I-IV and a pyridine containing compound, e.g., a compound with a similar structure to nicotine and/or cotinine.

### Methods

In another aspect, disclosed is a method of detecting and/or quantifying nicotine and/or cotinine in a sample. Also disclosed are methods of detecting and/or quantifying pyridine-containing compounds, e.g., a compounds with a similar structure to nicotine and/or cotinine. The sample can be any solid, gas, or liquid sample. In specific examples, the sample is a biological sample, such as urine, saliva, or blood sample. In other examples, the sample can contain one or more of Na⁺, K⁺, Ca²⁺, Mg²⁺, NH⁴⁺, Cl⁻, F⁻, alanine, arginine, leucine, bovine serum albumin (BSA), glucose, cholesterol, urea, uric acid, pyridine, and pyrrolidine. In other examples, the sample can be an environmental sample (e.g., water, gas, soil). The water sample can be a waste water, drinking water, black water, or grey water. The solid sample can be a soil sample or a clothing sample. In still other examples, the environmental sample can be an air sample (e.g., indoor air, outdoor air, air from an HVAC duct, air inside a vehicle, and the like.)

In the disclosed methods, the sample is contacted with any of the compounds disclosed herein, e.g., compounds of any one of Formulas I-IV. In a specific example, the sample is contacted with a compound of Formula I. In a preferred example, the sample is contacted with BiP-Am.

In the disclosed methods, the photoluminescence of the sample contacted with the compound of Formulas I-IV is measured. When nicotine and/or cotinine is present in the sample, the photoluminescence signal is quenched. Therefore, by comparing the photoluminescence signal prior to adding the sample or to a control sample without nicotine, a different signal can indicate the presence of nicotine and/or cotinine.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation can be required to optimize such process conditions.

All reagents and chemicals for synthesis were used without further purification. [1,1′-Biphenyl]-4,4′-dicarbaldehyde (97.08%) was purchased from AmBeed. Tris(2-aminoethyl)amine (98%) was purchased from TCI. Ammonium chloride, urea, sodium chloride, potassium chloride, magnesium sulfate, tetrabutylammonium chloride, tetrabutylammonium fluoride, acetone, acetonitrile, methanol, and dimethyl sulfoxide (DMSO) were provided by Fisher Chemical. Sodium chlorite was acquired from Acros Organics. (S)-(−)-Nicotine (99%) was purchased from Alfa Aesar. (1S)-(−)-α-pinene (98%), alpha-Cyano-4-hydroxycinnamic acid, THF, ethyl alcohol (99.5%), and uric acid were acquired from Thermo Scientific. Bovine Albumin serum (BSA) and cholesterol were provided from Gibco and MP Biomedicals, respectively. Calcium chloride, pyridine, pyrrolidine, L-Alanine, L-Arginine, L-Leucine, and glucose were purchased from Sigma Aldrich.

All NMR measurements were performed using a Bruker Neo 600 MHz NMR spectrometer.

The mass spectroscopy data were acquired on Bruker UltraFleXtreme MALDI-TOF/TOF. Ultra-pure alpha-Cyano-4-hydroxycinnamic acid matrix was used for sample investigations.

Thermogravimetric Analysis (TGA) measurements were performed on a TA Instruments Q50 Thermogravimetric Analyzer. About 9 mg sample was placed on an Alumina pan and heated up to 700° C. while the heating ramp was kept 5° C./minute under an inert atmosphere.

UV-Vis spectra were recorded on Shimadzu UV-1900i.

PL and TRPL studies were performed using an Edinburgh Instruments FS5 spectrofluorometer equipped with a 150 W xenon lamp and a 300 nm EPLED picosecond pulsed LED.

FS5 Spectrofluorometer equipped with an integrating sphere for photoluminescence quantum yield (PLQY) determination was utilized to measure quantum yield.

Scanning electron microscopy (SEM) measurements were performed using a high-resolution thermal field emission Hitachi SU-70 microscope at an accelerating voltage of 20 kV. Two different samples were prepared: one without nicotine and one with nicotine. For the sample without nicotine, 200 μL of a 1 mM BiP-Am solution was mixed with 800 μL of deionized water. For the sample with nicotine, 50 μL of a nicotine stock solution (0.1 M) was added to the same BiP-Am and deionized water mixture.

For the UV-Vis and fluorescence titrations, a 1 mM stock solution of BiP-Am was prepared in DMSO. The nicotine stock solution (0.1 M) was prepared by dissolving 16 μL of S-nicotine in 984 mL of distilled water and was further serially diluted to obtain the desired concentrations. A 10 UM concentration of BiP-Am (30 μL of 1 mM BiP-Am stock solution in total 3 mL aqueous sample) was used for each titration. For testing interfering analytes (Na⁺, K⁺, Ca²⁺, Mg²⁺, NH₄⁺, Cl⁻, F⁻, alanine, arginine, leucine, bovine serum albumin (BSA), glucose, cholesterol, urea, uric acid, and nicotine), standard solutions were prepared with a 0.1 M stock solution of each interferes and diluted to appropriate concentrations ranging from 0 to 1.0 mM for spectral recording. In the titration experiments, 3 mL of a BiP-Am solution (30 μL of probe in 2970 μL of distilled water) was placed in a quartz cuvette (path length 1 cm), and spectra were recorded after the addition of the appropriate analyte.

To determine the amount of nicotine in commercially available cigarettes, 250 mg of tobacco leaves were measured and ground to a powder. Subsequently, 5 mL of deionized (DI) water was added to extract the nicotine. The mixture was sonicated for 3 hours, then centrifuged and filtered to obtain a clear nicotine solution. The solution was diluted 100-fold with DI water. Urine samples were also diluted 100-fold and sonicated for 1 hour. The prepared solutions were then filtered and used for further analysis. Standard nicotine solutions were spiked into the cigarette and urine samples and quantified using a calibration curve.

The human urine samples were de-identified urine samples from persons 18-50 years of age. Corresponding IRB protocol #STUDY007287 was evaluated by the University IRB committee, concluding that: “The IRB determined that the proposed activity does not constitute research involving human subjects as defined by DHHS and FDA regulations.” Therefore, no written consent was required.

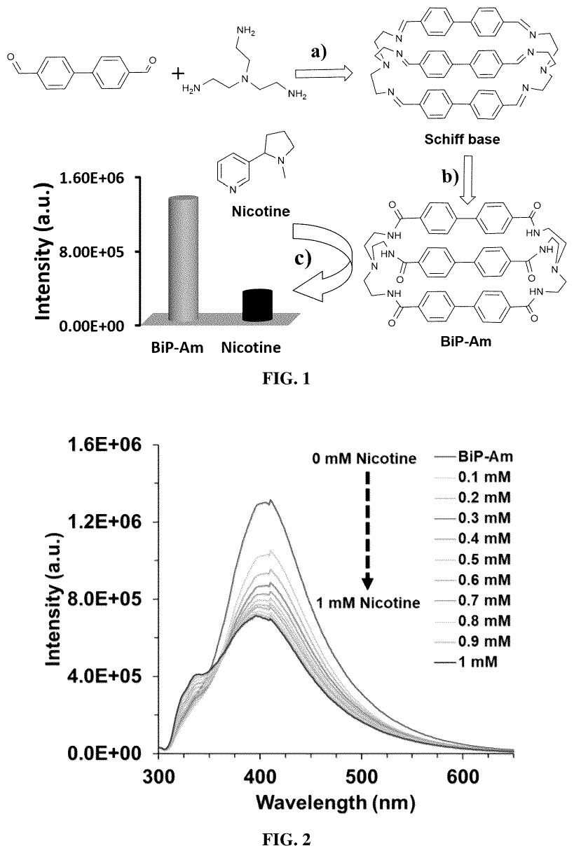

BiP-Am was synthesized via the Pinnick oxidation of the Schiff base (FIG. 1) (M. Boiocchi, et al., Angew. Chem. Int. Ed., 2004, 43, 3847-3852). The chemical structure and purity of the BiP-Am was confirmed by various analytical techniques such as NMR and MALDI-TOF.

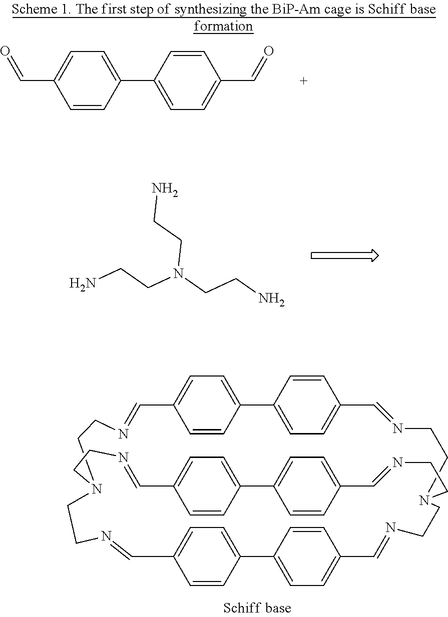 [SMILES: O=Cc1ccc(-c2ccc(C=O)cc2)cc1 ; NCCN(CCN)CCN ; C1=N/CCN2CC/N=C/c3ccc(cc3)-c3ccc(cc3)/C=N/CCN(CC/N=C/c3ccc(cc3)-c3ccc/1cc3)CC/N=C/c1ccc(cc1)-c1ccc(cc1)/C=N/CC2]

Specifically, to synthesize the BiP-Am cage, Schiff base formation (M. Boiocchi, et al., Angew. Chem. Int. Ed., 2004, 43, 3847-3852) and Pinnick oxidation techniques were employed. In the first step, 1 g of [1,1′-Biphenyl]-4,4′-dicarbaldehyde was dissolved in 150 mL of dry acetonitrile under inert conditions. The temperature was maintained at 0° C., and a solution of 470 μL Tris(2-aminoethyl)amine in 125 mL of dry acetonitrile was added dropwise over two hours under vigorous stirring and inert conditions. The reaction temperature was kept below 10° C. for 8 hours, followed by room temperature for 7 days. The products were then separated from the solution by vacuum filtration (1102 mg, 85%).

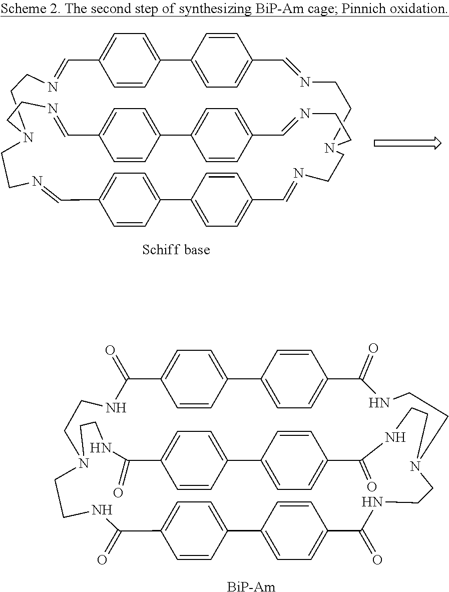 [SMILES: C1=N/CCN2CC/N=C/c3ccc(cc3)-c3ccc(cc3)/C=N/CCN(CC/N=C/c3ccc(cc3)-c3ccc/1cc3)CC/N=C/c1ccc(cc1)-c1ccc(cc1)/C=N/CC2 ; O=C1NCCN2CCNC(=O)c3ccc(cc3)-c3ccc(cc3)C(=O)NCCN(CCNC(=O)c3ccc(cc3)-c3ccc1cc3)CCNC(=O)c1ccc(cc1)-c1ccc(cc1)C(=O)NCC2 ; "BiP-Am"]

In the second step, 0.6 mmol of the synthesized Schiff base was suspended in 100 mL of dry THF under inert conditions. To this suspension, 20.1 mmol of sodium chlorite, 7.4 mmol of ammonium chloride, and 36.8 mmol of (1S)-(−)-α-pinene were added. The solution was then kept under reflux for 48 hours to obtain the white BiP-Am product. The synthesized BiP-Am was filtered and washed sequentially with methanol, water, and acetone, and then stored in a desiccator for drying (463 mg, 73%). ¹H NMR (600 MHZ, DMSO-d₆): δ_{H} ppm=2.65-2.76 (m, 12H) 3.48-3.57 (m, 12H) 7.16 (d, J=8.17 Hz, 12H) 7.44 (d, J=8.17 Hz, 12H) 7.73 (br t, J=4.81 Hz, 6H); ¹³C NMR (151 MHZ, DMSO-d₆): δ_{C} ppm=35.86, 50.56, 126.05, 127.22, 133.00, 140.66, 165.91; [M+H]⁺ ion=m/z=911.421.

Following the confirmation of the chemical structure of the probe molecule, its optical properties were evaluated. The absorption and emission spectra of the BiP-Am cage were examined in the presence of various solvents, such as tetrahydrofuran (THF), dichloromethane (DCM), acetonitrile (ACN), and methanol (MeOH) at room temperature. The absorption spectra (FIG. 5A) show that polar protic solvents can stabilize the BiP-Am probe in its ground state. The emission spectra recorded upon varying the solvent polarity under excitation at 273 nm (FIG. 5B) revealed that polar protic solvents such as MeOH can cause significant quenching along with a blue shift in the emission spectra in comparison with polar aprotic solvents such as DCM (E. Fresch, et al., Molecules, 2023, 28, 3553).

Upon successive addition of water fractions, up to 99% in a solution containing BiP-Am in DMSO, an increase in the absorbance intensity is observed along with a slight red shift of the recorded spectra (FIG. 6A). As depicted in FIG. 6B, the emission spectrum of the BiP-Am in H₂O:DMSO (9.9:0.1, v:v, 10 μM) exhibited an intensity enhancement along with a blue shift from 426 nm in pure DMSO (Φ=2.13%) to 410 nm in H₂O:DMSO (9.9:0.1, v:v, 10 μM) (Φ=5.46%) at an excitation wavelength of 273 nm, which indicates the formation of aggregation-induced emission enhancement (AIEE).

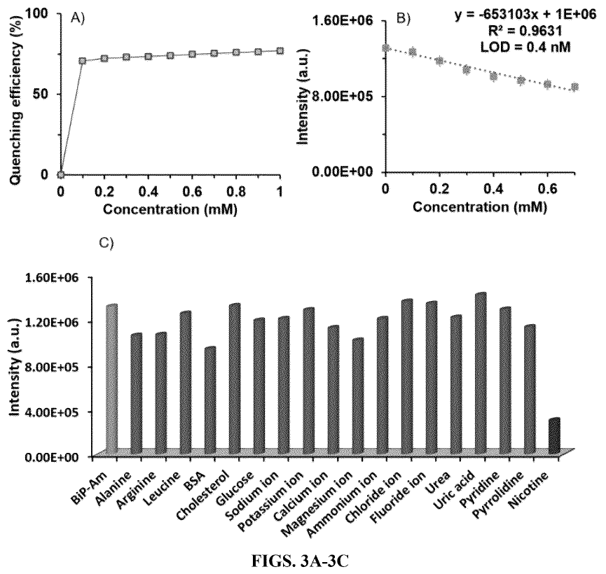

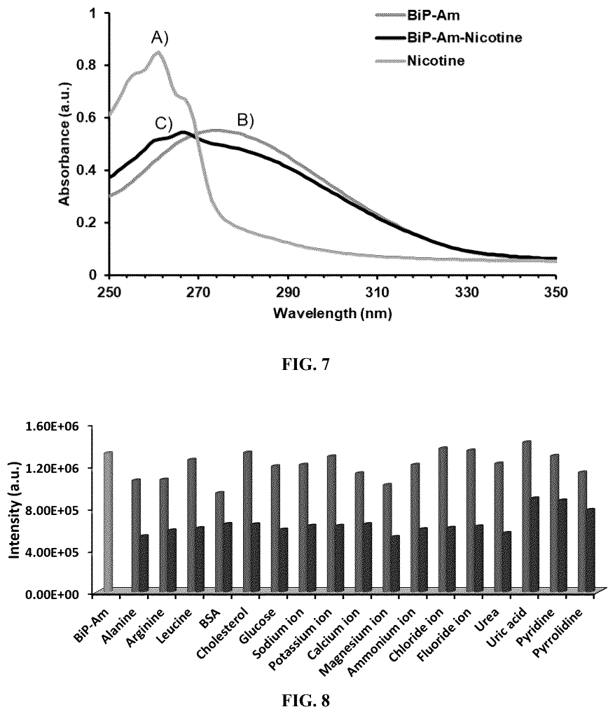

Upon addition of nicotine (1 mM) to a BiP-Am solution (H₂O:DMSO, 9.9:0.1, v:v, 10 μM), the absorption intensity of the probe decreases along with the appearance of a red-shifted absorption band of nicotine at 265 nm (FIG. 7). As shown in FIG. 2, photoluminescence (PL) studies revealed an acceptable quenching (Φ=3.04%) upon the addition of nicotine (1 mM) into the H₂O:DMSO (9.9:0.1, v:v, 10 μM) solution of the BiP-Am, with a quenching efficiency of 77% (FIG. 3A). Quenching efficacy was calculated by using

$$Q E = \frac{1 - F}{F 0}$$

formula; F₀ represents the fluorescence intensity measured prior to the addition of nicotine to the H₂O:DMSO (9.9:0.1, v:v, 10 μM) solution, while F represents the fluorescence intensity measured after nicotine is added.

In FIG. 2, the effect of different nicotine concentrations on the BiP-Am probe emission spectra is presented. The limit of detection (LOD) was calculated using the formula

$$LOD = \frac{3 σ}{m} ,$$

where σ represents the standard deviation of the signal and m denotes the slope of the calibration curve. The LOD was determined to be approximately 0.4 nM (when σ is 0.008), demonstrating the probe's excellent sensitivity for detecting nicotine in diluted samples (FIG. 3B). Notably, this is the best-reported value in the literature using fluorescence as a detection method and second best overall.

Selectivity studies of BiP-Am probes were performed in the presence of potential interfering analytes such as Na⁺, K⁺, Ca²⁺, Mg²⁺, NH⁴⁺, Cl⁻, F⁻, alanine, arginine, leucine, bovine serum albumin (BSA), glucose, cholesterol, urea, uric acid, pyridine, pyrrolidine, and nicotine. The concentration of all interferents was 1 mM and a negligible quenching effect was observed in the presence of these analytes except for nicotine (FIG. 3C). This performance demonstrated the excellent selectivity of the probe molecule and its potential for utilization in real-world samples.

Toward this end, for real-time evaluation of BiP-Am in biological samples such as urine, competitive experiments were carried out in the presence of all the above-mentioned interfering analytes by setting their concentration to 1 mM, in the presence of 1 mM nicotine. As shown in FIG. 8, no significant change is observed in the presence of other interfering analytes, indicating the strong selectivity of the probe towards nicotine.

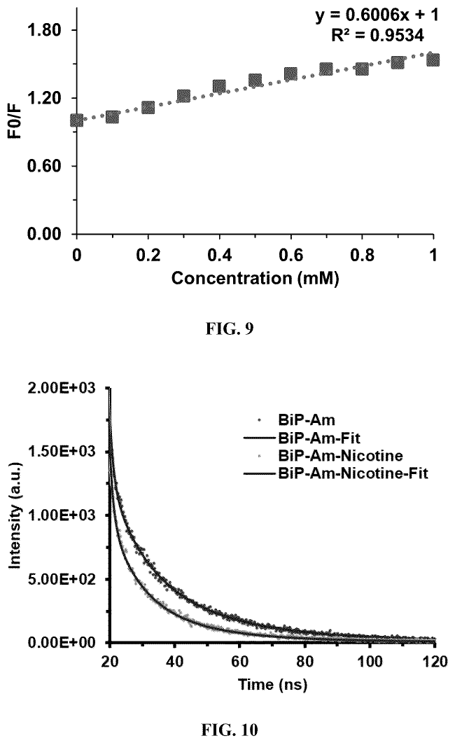

A Stern-Volmer diagram is depicted in FIG. 9, showing the ratio of sample PL intensity before and after the addition of the quencher versus the quencher's concentration, F₀/F=K [Q]+1. Here, F₀ is the fluorescence intensity before the addition of nicotine to the solution H₂O:DMSO (9.9:0.1, v:v, 10 μM), and F is the fluorescence after the addition of nicotine, K is the quenching constant, and [Q] is the quencher's concentration. The linear dependence (R²=0.953) of the ratio F₀/F on the quencher concentration indicates the dynamic nature of quenching. The slope of the linear regression between F₀/F and [nicotine] gave a value of K=0.6 mM-1.

To evaluate the excited state interactions, time-resolved photoluminescence (TRPL) studies were carried out with an excitation wavelength of 300 nm (FIG. 10) (Q. Zhang, et al., Nanoscale, 2020, 12, 1826-1832). The time-resolved studies of BiP-Am in H₂O:DMSO (9.9:0.1, v:v, 10 μM) showed an amplitude-weighted average lifetime (<τ>ₐₘₚ) and an intensity-weighted average lifetime (<τ>ᵢₙₜ) of 6 ns and 19 ns, respectively (τ₁=1.22 ns, 14.87%; τ₂=10.11 ns, 22.61%; τ₃=26.47 ns, 62.52%). Upon addition of nicotine to the BiP-Am solution, there was a significant decrease in both (T) amp and (T) int to 4 ns and 13 ns, respectively (τ₁=1.19 ns, 23.84%; τ₂=9.31 ns, 34.44%; τ₃=23.51 ns, 41.72%), indicating that the quenching mechanism is primarily dynamic.

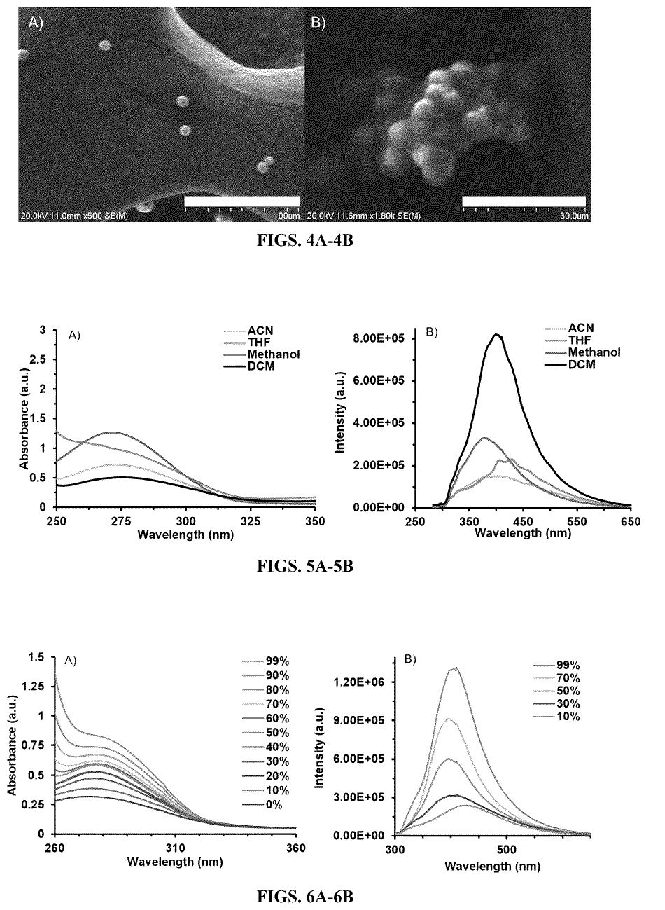

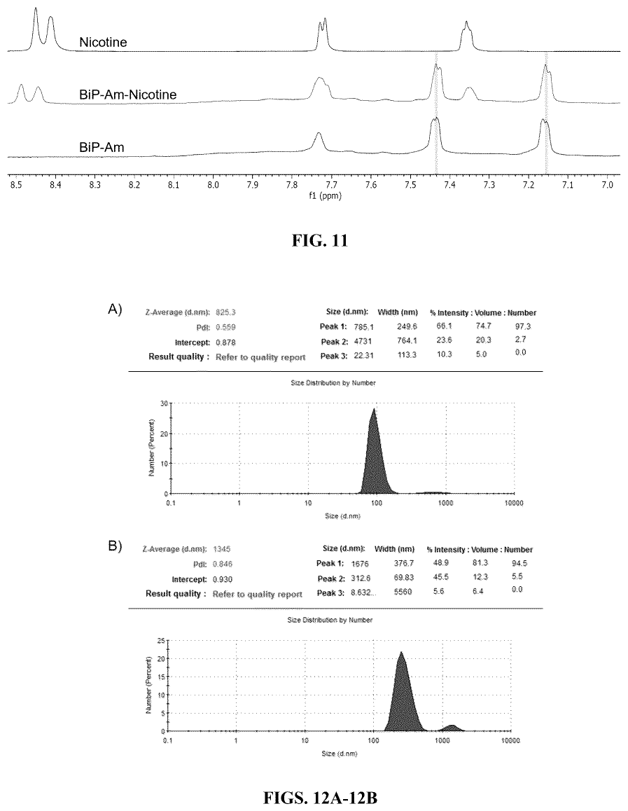

Moreover, to get an insight into the molecular interaction between nicotine and BiP-Am, ¹H NMR studies in DMSO-d₆:D₂O (9:1, v:v) were carried out. It was revealed that the aromatic protons of the BiP-Am exhibited a slight upfield shift (0.008 ppm) (FIG. 11). This indicates potential nicotine-induced enhanced π-π or hydrogen bond interactions of the analyte with the probe molecules. Furthermore, dynamic light scattering (DLS) results revealed an aggregation upon the addition of nicotine to the BiP-Am sample (H₂O:DMSO (9.9:0.1, v:v)), while the particle size changed from 825 nm to 1345 nm (FIGS. 12A-12B). The induced aggregation upon the addition of nicotine solution to the BiP-Am sample was analyzed by scanning electron microscopy (SEM). The BiP-Am probe SEM image in FIG. 4A shows dispersed spherical-shaped particles, which form closely packed aggregates in the presence of nicotine (FIG. 4B), corroborating further the strong interaction among the probe molecules in the presence of the analyte.

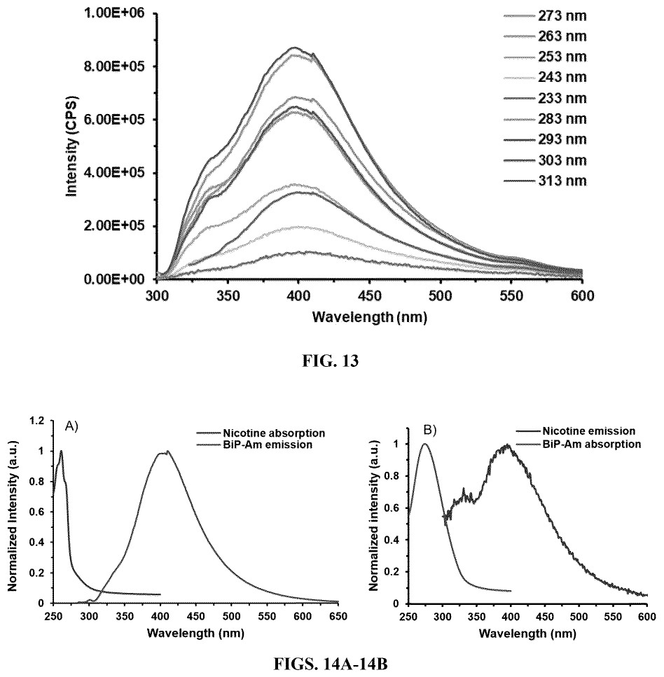

In order to demonstrate the mechanism of action in the detection of nicotine by the probe molecules, the following experiments were performed. The emission spectra of the BiP-Am probe in the presence of nicotine at different excitation wavelengths, from 233 nm to 313 nm, were carried out. It was observed that by changing the excitation wavelength, a variation in emission intensity was recorded, while no change in λₘₐₓ was detected (FIG. 13), indicating an IFE mechanism of action (P. Sharma, et al., ACS Omega, 2020, 5, 19654-19660).

The absorption spectrum of nicotine recorded in H₂O:DMSO (9.9:0.1, v:v, 10 μM) displayed a narrow absorption band from 250-320 nm with \tilde{λ}ₘₐₓ at 260 nm, which barely overlaps with the emission spectrum of probe BiP-Am ranging from 300 nm-650 nm with \tilde{λ}ₘₐₓ at 410 nm (FIG. 14A). Simultaneously, upon analysis, the emission spectrum of nicotine, which ranges from below 300 to 600 nm slightly overlaps with the BiP-Am probe absorption spectrum 250-360 nm with \tilde{λ}ₘₐₓ at 273 nm (FIG. 14B). These results rule out FRET as a possible mechanism in this system.

TABLE 1
Nicotine detection in real world samples

| | Detected | Added | Found | STD | RSD |
|---|---|---|---|---|---|
| Sample | nicotine | nicotine | (mM) | (%) | (%) |
| Cigarette | 0.208 | | | 0.03 | 0.17 |
| Spike 1 | | 0.500 | 0.723 | 0.04 | 0.08 |
| Spike 2 | | 0.700 | 0.901 | 1.03 | 1.50 |
| Urine | Not | | | | |
| Spike 1 | | 0.300 | 0.326 | 0.45 | 1.49 |
| Spike 2 | | 0.500 | 0.512 | 0.27 | 0.54 |

Density Functional Theory (DFT) and Time-Dependent Density Functional Theory (TDDFT) calculations, employing the B3LYP functional with the 6-31G (d) basis set, were conducted to investigate the possibility of photo-induced electron transfer in the BiP-Am probe upon excitation. The results indicate that such a transfer is unlikely, as the excited-state HOMO energy level of BiP-Am (−5.029 eV) is higher than the ground-state HOMO energy level of nicotine (−5.508 eV). This energy difference indicates that electron transfer from Nicotine to BiP-Am is not energetically favorable.

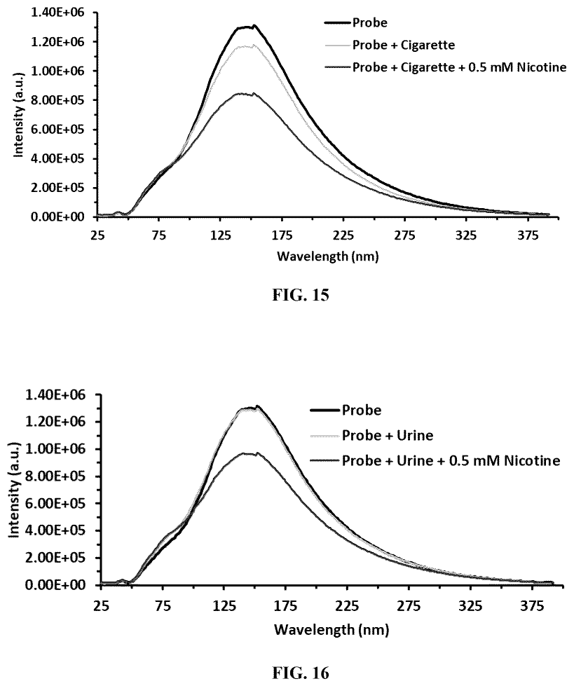

In order to demonstrate the potential of the disclosed probe molecule in real-world samples, BiP-Am was utilized for the detection of nicotine in human urine samples and extracted nicotine solutions from cigarettes. The cigarette samples were diluted in distilled water, followed by sonication, centrifugation, and filtration to obtain a clear solution. The nicotine content in cigarettes, calculated using the standard calibration method without spiking, was found to be 0.208 mM. The cigarette solution was further spiked with standard nicotine solution (0.500 and 0.700 mM) and calculated the amount of nicotine using the calibration curve. Examining closely the cigarette and 0.500 mM case, an amount of 0.723 mM was determined, almost identical to the total expected one (0.500+0.208=0.708). Apparently, the determined amount matched the actual nicotine amount pretty well, demonstrating the robustness of the method (FIG. 15).

Considering the human urine sample analysis, a 100-fold dilution was performed before the quantification of the nicotine content. The urine samples were also spiked with a known concentration of nicotine (0.300 and 0.500 mM), while a standard calibration curve was used to identify the spiked nicotine concentration (Table 1). Notably, the relative standard deviation was found to be less than 1.5%, while the determined amount matches exactly the spiked amount (e.g. 0.500 mM vs 0.512 mM, FIG. 16). These results indicate that the BiP-Am probe holds potential for the selective and efficient detection of nicotine in real-world samples, such as human urine and cigarettes. Further modifications and optimizations could enhance its performance and broaden its practical applications.

In conclusion, amide-based cages, e.g., BiP-Am, were designed, synthesized, and utilized as a probe to detect nicotine in an aqueous media with excellent selectivity and sensitivity. The corresponding detection limit lies in the nanomolar range, among the best reported in the literature, making it suitable for real-time applications. TRPL and DFT studies confirmed the dynamic quenching mechanism primarily due to the inner-filter effect of nicotine. Further validation through ¹H NMR studies, dynamic light scattering, and SEM measurements provided insight into the molecular interactions and aggregation behaviour of the BiP-Am probe in the presence of nicotine. The probe was effectively applied to detect nicotine in real samples, including cigarette extracts and human urine, showcasing its potential for practical applications. These findings indicate that BiP-Am and compounds of Formulas I-IV can serve as effective sensor for nicotine detection.

Other advantages, which are obvious and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. A compound of Formula I: 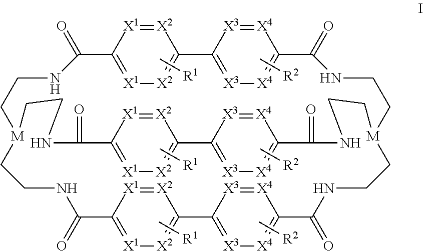
wherein
each X¹, X², X³, and X⁴ are, independently, CH, N, CR¹, or CR²;
R¹ and R² are, independently, H, OH, SH, C₁₋₆ alkyl, C₁₋₆ alkoxyl, C₁₋₆ thioalkyl C₁₋₆ alkyl-SO₄, F, Cl, Br, NH₂, NO₂, or CN; and
M is N or N⁺R⁴, wherein R⁴ is H or C₁₋₆ alkyl.

2. The compound of claim 1, wherein any one of X¹, X², X³, and X⁴ is N.

3. The compound of claim 1, wherein any two of X¹, X², X³, and X⁴ are N.

4. The compound of claim 1, wherein three of X¹, X², X³, and X⁴ are N.

5. The compound of claim 1, wherein all of X¹, X², X³, and X⁴ are CH.

6. The compound of claim 1, wherein one or more R¹ is OH, SH, C₁₋₆ alkyl, C₁₋₆ alkoxyl, C₁₋₆ thioalkyl, C₁₋₆ alkyl-SO₄, F, Cl, Br, NH₂, NO₂, or CN.

7. The compound of claim 1, wherein one or more R² is OH, SH, C₁₋₆ alkyl, C₁₋₆ alkoxyl, C₁₋₆ thioalkyl, C₁₋₆ alkyl-SO₄, F, Cl, Br, NH₂, NO₂, or CN.

8. The compound of claim 1, wherein each R¹ and R² are OH, SH, C₁₋₆ alkyl, C₁₋₆ alkoxyl, C₁₋₆ thioalkyl, C₁₋₆ alkyl-SO₄, F, Cl, Br, NH₂, NO₂, or CN.

9. The compound of claim 1, wherein each Mis N.

10. The compound of claim 1, wherein each Mis N⁺R⁴, with R⁴ being H or C₁-C₆ alkyl.

11. The compound of claim 1, wherein one M is N and the other M is N⁺R⁴, with R⁴ being H or C₁₋₆ alkyl.

12. The compound of claim 1, wherein the compound is:

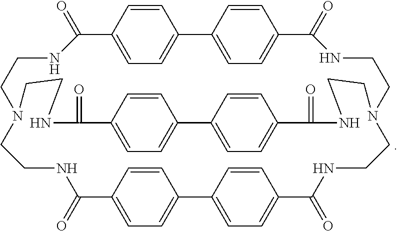 [SMILES: O=C1NCCN2CCNC(=O)c3ccc(cc3)-c3ccc(cc3)C(=O)NCCN(CCNC(=O)c3ccc(cc3)-c3ccc1cc3)CCNC(=O)c1ccc(cc1)-c1ccc(cc1)C(=O)NCC2]

13. A composition comprising the compound of claim 1 and nicotine or cotinine.

14. A method of detecting or quantifying nicotine and/or cotinine in a sample, comprising:
contacting the sample with the compound of claim 1; and
measuring photoluminescence in the sample,
wherein when nicotine and/or cotinine are present, the photoluminescence of the sample is reduced as compared to a control without nicotine and/or cotinine.

15. The method of claim 14, wherein the sample is a biological sample.

16. The method of claim 15, wherein the sample is a urine, saliva, or blood sample.

17. The method of claim 14, wherein the sample is an environmental sample.

18. The method of claim 17, wherein the environmental sample comprises a soil or water sample.

19. The method of claim 14, wherein the sample further comprises one or more of Na⁺, K⁺, Ca²⁺, Mg²⁺, NH⁴⁺, Cl⁻, F⁻, alanine, arginine, leucine, bovine serum albumin (BSA), glucose, cholesterol, urea, uric acid, pyridine, and pyrrolidine.

20. The method of claim 14, wherein the method detects nicotine.
